(19) United States
(12) Patent Application Publication
(10) Pub. No.: US 2026/0090702 A1
(43) Pub. Date: Apr. 2, 2026

(54) AUXILIARY CAP FOR ENDOSCOPE

(71) Applicant: ENDOROBOTICS CO., LTD., Seoul (KR)

(72) Inventors: Byung Gon KIM, Seoul (KR); Kyung Nam KIM, Seoul (KR)

(21) Appl. No.: 19/110,740

(22) PCT Filed: Sep. 6, 2023
(86) PCT No.: PCT/KR2023/013346

§ 371 Date: Mar. 11, 2025

(30) Foreign Application Priority Data
Sep. 14, 2022 (KR) 10-2022-0115510

(51) Int. Cl. A61B 1/00; A61B 17/29

(52) U.S. Cl. CPC A61B 1/00137; A61B 1/00177; A61B 17/29; A61B 2017/2902

(57) ABSTRACT

An auxiliary cap for an endoscope is coupled to an endoscopic device and includes a first housing including a housing body that can be coupled to the tip of the endoscopic device, and a first opening located at a position corresponding to a light source, forceps hole, and a camera module of the endoscope device when the housing body and the endoscopic device are coupled; an auxiliary procedure member including a flexible manipulator having a portion of the length that can be bent in one direction by force transmitted through a drive cable, and a gripper provided on the end side of the flexible manipulator to assist a procedure performed using the forceps; and a second housing formed on one side of the first housing to form an accommodation space for accommodating a portion, including the gripper, of the entire length of the auxiliary procedure member.

### TECHNICAL FIELD

The present invention relates to an auxiliary cap for an endoscope.

### BACKGROUND

Endoscopic retrograde cholangio pancreatography (ERCP) is a procedure that diagnoses and treats various diseases by examining the liver, bile ducts, gallbladder, and pancreas using an endoscopic device.

These endoscopic devices commonly used for ERCP are of the side-view type in which a light source and forceps are provided on the side of the main body.

That is, in the above-described side-view endoscopic device, a light source provides light to the side of the main body, and forceps for examination or procedure are also pulled out to the side of the main body through a forceps hole formed on the side of the main body.

Through this, the user can diagnose and treat diseases by inserting forceps into other organs located on the side of an organ such as the duodenum, such as the pancreas or bile duct, through the light provided on the side of the main body.

Accordingly, in order to easily insert the forceps into the pancreas or bile duct from inside the duodenum, the end of the forceps withdrawn laterally from the main body of the endoscopic device should be placed in a position aligned with the hole connecting the duodenum to the pancreas or bile duct.

Because of this, if the side of the main body from which the forceps are withdrawn and the hole connecting the duodenum to the pancreas or bile duct do not face each other, there is a problem that the difficulty of the procedure increases because it is difficult to enter the forceps withdrawn from the side of the main body into the hole.

Accordingly, conventional ERCP is a procedure that only highly skilled practitioners can perform, which increases the barrier to entry for the procedure.

### DISCLOSURE

### Technical Problem

The present invention is to solve the above problems, and the present invention is directed to providing an auxiliary cap for an endoscope capable of assisting in the procedure of forceps.

The present invention is also directed to providing an auxiliary cap for an endoscope capable of securing a forward field of vision.

The problems of the present invention are not limited to those mentioned above, and other problems not mentioned will be clearly understood by those of ordinary skill in the art from the following description.

### Technical Solution

According to one aspect of the present invention, provided is an auxiliary cap for an endoscope, the auxiliary cap is coupled to an endoscopic device including a light source, a forceps hole, and a camera module, and the auxiliary cap includes a first housing comprising a housing body, which is formed in the shape of an enclosure and has an internal space with an open lower portion so that the housing body can be coupled to the tip of the endoscopic device, and a first opening, which has a predetermined area and passes through the housing body so as to be located at a position corresponding to the light source, the forceps hole, and the camera module when the housing body and the endoscopic device are coupled; an auxiliary procedure member comprising a flexible manipulator, which is provided so that a portion of the length can be bent in one direction by external force transmitted through a drive cable, and a gripper, which is provided on the end side of the flexible manipulator so as to be able to assist a procedure performed using forceps; and a second housing formed on one side of the first housing so as to form an accommodation space for accommodating a portion, including the gripper, of the entire length of the auxiliary procedure member.

In addition, the endoscopic device may be a side-view endoscopic device in which the light source, the forceps hole, and the camera module are provided on the lateral side of a tip of a main body, and the first opening may be formed to be positioned on the lateral side of the housing body.

In addition, the second housing may include a second opening formed to communicate with the accommodation space so that the gripper accommodated in the accommodation space is able to move from the accommodation space to the outside of the second housing.

In addition, the second opening may be formed in the second housing to be positioned in the same direction as the first opening.

In addition, the flexible manipulator may include a hollow body having a predetermined length, a plurality of first cut-out portions formed circumferentially along the body, a plurality of second cut-out portions formed circumferentially along the body without being connected to the first cut-out portions, and a plurality of drive cables, each of which is fixed to an end side of the body.

In addition, the auxiliary cap for an endoscope may further include an auxiliary light source and an auxiliary camera module provided at the tip of the first housing.

### ADVANTAGEOUS EFFECT

According to the above configuration, the auxiliary cap for an endoscope according to the present invention can be combined with the tip of a conventional endoscopic device to assist in a procedure with forceps, thereby reducing the difficulty of the procedure.

In addition, the auxiliary cap for an endoscope according to the present invention can secure a forward field of vision, thereby reducing the difficulty of the procedure.

Advantageous effects of the present invention are not limited to the above-described effects, and should be understood to include all effects that can be inferred from the configuration of the invention described in the detailed description or claims of the present invention.

### BRIEF DESCRIPTION OF THE DRAWINGS

FIG. 1 is a drawing showing a state in which an auxiliary cap for an endoscope according to an example embodiment of the present invention is coupled to a tip of an endoscopic device.

FIG. 2 is a drawing showing a state in which the auxiliary cap for an endoscope according to an example embodiment of the present invention is separated from the endoscopic device in FIG. 1.

FIG. 3 is a drawing showing an auxiliary cap for an endoscope according to an example embodiment of the present invention.

FIG. 4 is a schematic diagram showing a flexible manipulator that can be applied to an auxiliary procedure member of an auxiliary cap for an endoscope according to an example embodiment of the present invention.

FIG. 5 is a drawing showing a state in which a part of an auxiliary procedure member is bent in an auxiliary cap for an endoscope according to an example embodiment of the present invention.

FIG. 6 is a drawing showing another form of an auxiliary cap for an endoscope according to an example embodiment of the present invention.

FIG. 7 is a use state diagram of an auxiliary cap for an endoscope according to an example embodiment of the present invention.

### MODES OF THE INVENTION

Hereinafter, example embodiments of the present invention will be described in detail so that those of ordinary skill in the art can readily implement the present invention with reference to the accompanying drawings. The present invention may be embodied in many different forms and is not limited to the embodiments set forth herein. In the drawings, parts unrelated to the description are omitted for clarity of description of the present invention, and throughout the specification, same or similar reference numerals denote same elements.

Terms and words used in the present specification and claims should not be construed as limited to their usual or dictionary definition. They should be interpreted as meaning and concepts consistent with the technical idea of the present invention, based on the principle that inventors may appropriately define the terms and concepts to describe their own invention in the best way.

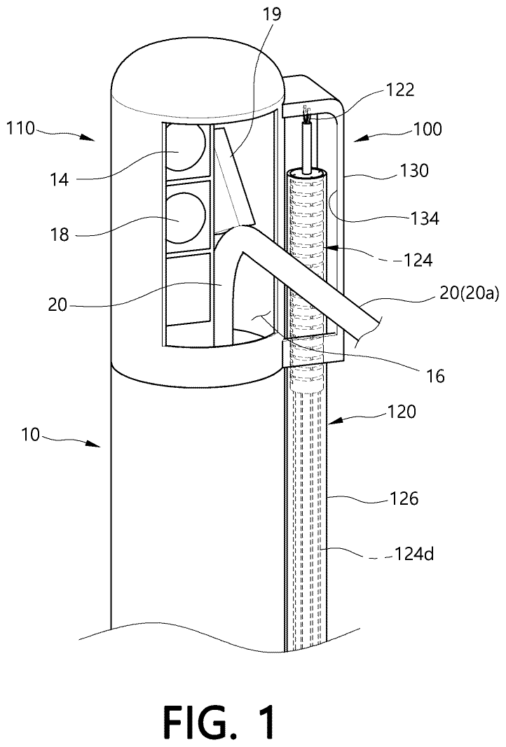

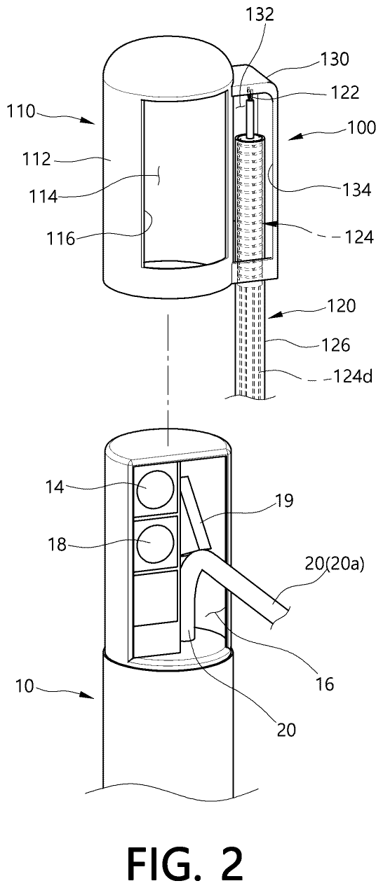

An auxiliary cap 100 for an endoscope according to an example embodiment of the present invention can be detachably coupled to a tip of an endoscopic device 10 as shown in FIGS. 1 and 2.

For example, as shown in FIG. 2, the endoscopic device 10 may be a known side-view endoscopic device, including a light source 14 that provides light to a lateral side of a tip of a main body 12, a forceps hole 16 into which forceps 20 are inserted for performing a tissue examination or other procedure, a camera module 18 for receiving image information, and a manipulation member 19 rotatably positioned in the forceps hole 16 so that the direction of the forceps 20 accommodated in the forceps hole 16 can be changed when the user manipulates it.

That is, the side-view endoscopic device may emit light generated from the light source 14 provided on the lateral side of the tip of the main body 12 in a direction perpendicular to the longitudinal direction of the main body 12.

In addition, the forceps 20 may be a surgical tool or a procedural tool, such as a commonly known stent, grasper, catheter, cannula, used in a conventional endoscope for purposes such as supporting, holding, or pressing tissue or organs, as a tool used in surgery or treatment in the surgical field.

In the present invention, the side-view endoscopic device may be an endoscopic device used for ERCP (Endoscopic Retrograde Cholangio Pancreatography) to examine the liver, bile duct, gallbladder, and pancreas to diagnose and treat diseases, and may be of a type that emits light generated from a light source to the lateral side of the main body, and the forceps for examination or treatment may also be pulled out to the side of the endoscopic device through the manipulation member 19 from the forceps hole.

In this case, the auxiliary cap 100 for an endoscope according to an example embodiment of the present invention may be coupled to the tip of the endoscopic device 10 as described above.

Accordingly, the auxiliary cap 100 for an endoscope according to an example embodiment of the present invention has the advantage of being able to use an existing endoscopic device as is since it can be detachably coupled to the tip of a known endoscopic device 10 without changing the structure of the known endoscopic device 10.

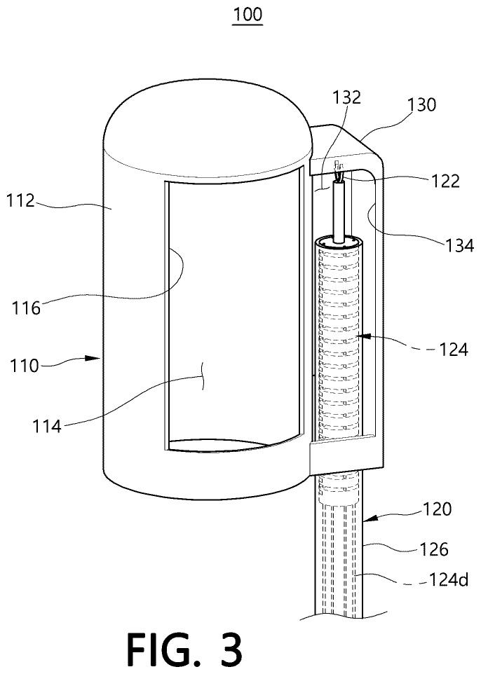

To this end, the auxiliary cap 100 of an endoscope according to an example embodiment of the present invention may include a first housing 110, an auxiliary procedure member 120, and a second housing 130 as shown in FIG. 3.

The first housing 110 may be formed in an enclosure shape having an internal space 114 with an open lower portion, and may include a first opening 116 penetrating a predetermined area at positions corresponding to the light source 14, the forceps hole 16, and the camera module 18.

Here, the internal space 114 may be a space for accommodating the tip of the endoscopic device 10, that is, a portion of the length of the main body 12, and the open lower portion of the first housing 110 may serve as a coupling hole for coupling with the tip of the main body 12.

Accordingly, the first housing 110 may be detachably coupled to the tip of the endoscopic device 10, i.e., the tip of the main body 12, through the open lower portion, and the first opening 116 may be placed at a position corresponding to the light source 14, the forceps hole 16, and the camera module 18 provided in the main body 12 when the first housing 110 and the main body 12 are coupled.

Through this, light irradiated from the light source 14 may be emitted to the outside through the first opening 116, and a portion of the total length of the forceps 20 placed within the forceps hole 16 may be drawn out to the outside through the first opening 116.

In this case, the camera module 18 provided in the main body 12 may obtain an image of an object located outside the first opening 116 using light emitted from the light source 14, and the image acquired through the camera module 18 may be transmitted to a display (not shown) of the endoscopic device 10.

Here, the shape of the object may be the inside of an organ such as the esophagus, stomach, small intestine, duodenum, liver, bile duct, gallbladder, pancreas, etc.

That is, the first opening 116 may serve as an emission path that emits light emitted from the main body 12 to the outside and as a working channel through which a portion of the forceps 20 protruding a certain length from the forceps hole 16 of the main body 12 passes.

Through this, even though the auxiliary cap 100 for an endoscope according to an example embodiment of the present invention is coupled to the tip of a conventional endoscopic device 10, a portion of the length of the forceps 20 may protrude outside the first housing 110 through the first opening 116, thereby allowing surgery or procedure to be performed in the same manner as in the conventional method.

For example, when the auxiliary cap 100 for an endoscope according to an example embodiment of the present invention is applied to a side-view endoscopic device 10 as shown in FIGS. 1 and 2, the first housing 110 may include a housing body 112 formed in a hollow cylindrical shape with a sealed upper portion and an open lower portion, and a first opening 116 formed through a predetermined area in the housing body 112, and the first opening 116 may be formed on a lateral side of the housing body 112.

In this case, the open lower portion of the housing body 112 may have approximately the same size as the tip of the main body 12.

Accordingly, the first housing 110 may be coupled to the tip of the main body 12 through the open lower portion of the housing body 112 in a force-fitting manner, and even though the auxiliary cap 100 for an endoscope according to an example embodiment of the present invention is coupled to the tip of a conventional side-view endoscopic device 10, a bent portion 20a of the forceps 20 may protrude outside the first housing 110 through the first opening 116, thereby allowing surgery or procedure to be performed in the same manner as in the conventional method.

However, the shape of the first housing 110 is not limited to this, and if it has a shape that is detachably coupled to the tip of the main body 12 and has an internal space 114, it may be appropriately changed to fit the shape of the main body 12, and the formation position of the first opening 116 may be appropriately changed depending on the positions of the light source 14, the forceps hole 16, and the camera module 18 provided in the endoscopic device.

The auxiliary procedure member 120 may assist in a procedure using the forceps 20 drawn out of the first housing 110 through the first opening 116.

That is, the auxiliary procedure member 120 may be provided so that a portion of the total length may be bent, and may include a gripper 122 for assisting in a procedure using the forceps 20.

For example, when the endoscopic device is provided as a side-view endoscopic device, the auxiliary procedure member 120 may be provided to be bent in the same direction as the bent portion 20a of the forceps 20 that is bent through the manipulation member 19.

Accordingly, the auxiliary procedure member 120 may identify an entry hole 30 of an organ 40 by grasping the inner wall of the organ using the gripper 122 while being bent in the same direction as the bent portion 20a of the forceps 20, or may expand the size of the entry hole 30 by pulling the tissue around the entry hole 30 of the organ 40.

Through this, the bent portion 20a of the forceps 20 may easily enter the interior of the organ 40 through the entry hole 30 of the organ 40.

However, the role of the gripper 122 is not limited to this, and it may cut and collect a part of the tissue, and may perform all the roles performed by the known endoscopic gripper used in endoscopic procedures.

To this end, the auxiliary procedure member 120 may include a flexible manipulator 124, which is provided so that a portion of the length can be bent in one direction by external force transmitted through a drive cable 124d, and a gripper 122 provided on the end side of the flexible manipulator 124 to assist in the procedure using the forceps 20.

For example, the flexible manipulator 124 may be a known continuum manipulator, and the gripper 122 may be a known a gripper for an endoscope.

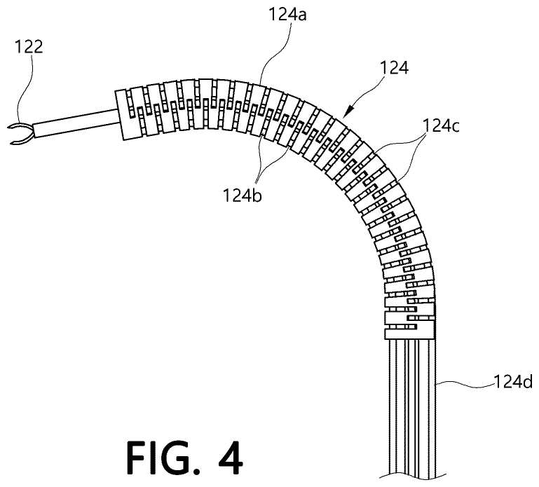

As a non-limiting example, as shown in FIG. 4, the flexible manipulator (124) may include a hollow body 124a having a predetermined length, a plurality of first cut-out portions 124b formed circumferentially along the body 124a, a plurality of second cut-out portions 124c formed circumferentially along the body 124a without being connected to the first cut-out portions 124b, and a plurality of drive cables 124d, each of which is fixed to an end side of the body 124a.

In this case, the tip of the body 124a may be sealed, one end of each of the plurality of drive cables 124d may be fixed to the sealed tip, and the plurality of driving cables 124d, with each end fixed to the sealed tip, respectively, may be arranged along an edge of the sealed tip and spaced apart from each other at a distance.

In addition, the plurality of first cut-out portions 124b and the plurality of second cut-out portions 124c may be formed on the circumferential surface of the body 124a so as to be alternately arranged along the longitudinal direction of the body 124a.

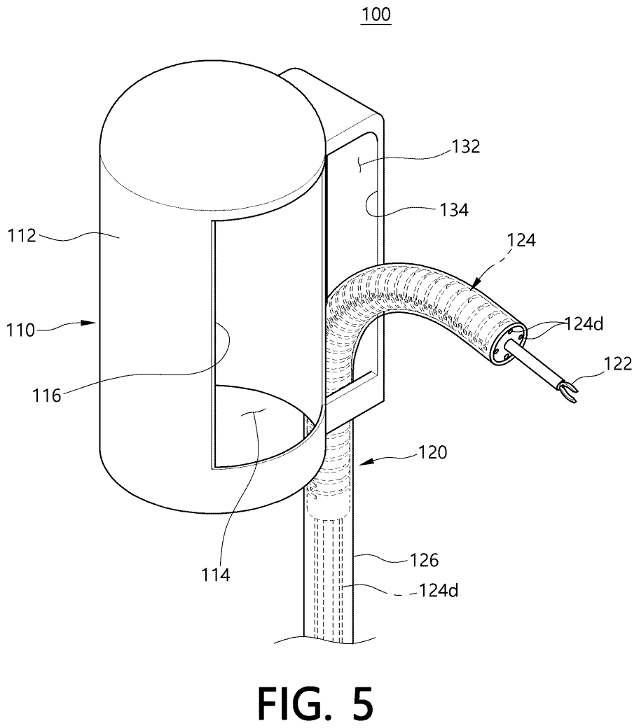

Accordingly, when a user manipulates at least one of the plurality of drive cables 124d, the flexible manipulator 124 may bend in one direction a portion of its total length using the plurality of first cut-out portions 124b and/or the plurality of second cut-out portions 124c, as shown in FIGS. 4 and 5.

However, the detailed configuration of the flexible manipulator 124 applicable to the present invention is not limited thereto, and various known continuum manipulators may be configured as the flexible manipulator described above.

In this case, the gripper 122 may be provided so as to be exposed to the outside at the sealed tip, which is the tip of the body 124a.

In addition, the gripper 122 may be provided to be positioned at the tip of the body 124a, may be provided to protrude a certain length from the tip of the body 124a along the longitudinal direction of the body 124a, and may be provided at the tip of the body 124a so as to be able to move along a direction parallel to the longitudinal direction of the body 124a.

Accordingly, when a portion of the length of the flexible manipulator 124 is bent in one direction by the plurality of drive cables 124d, the gripper 122 provided at the tip of the body 124a may change position along with the bent portion of the length of the flexible manipulator 124

Here, the auxiliary procedure member 120 may further include a hollow tube 126 that surrounds the flexible manipulator 124, and the plurality of drive cables 124d each having one end fixed to the tip of the body 124a may be arranged to be positioned inside the tube 126.

In addition, a manipulation cable (not shown) for manipulating the operation of the gripper 122 may also be arranged to be positioned inside the tube 126.

In addition, the tube 126 may be provided to wrap the entire length of the flexible manipulator 124, or may be provided to wrap only a portion of the entire length of the flexible manipulator 124.

The second housing 130 may be formed on one side of the first housing 110 so as to form an accommodation space 132 that accommodates a portion of the total length of the auxiliary procedure member 120.

That is, the second housing 130 may be formed in an enclosure shape having an accommodation space 132 with an open lower portion.

Accordingly, some lengths including the gripper 122 of the auxiliary procedure member 120 may enter the accommodation space 132 through the open lower portion of the accommodation space 132.

Here, the second housing 130 may be integrally formed with the first housing 110, or may be formed as a separate member to be coupled to one side of the first housing 110.

In addition, the accommodation space 132 may be formed in the second housing 130 to accommodate a portion of the length of the flexible manipulator 124 that bends in one direction by the manipulation of the drive cables 124d along with the gripper 122 among the total length of the auxiliary procedure member 120.

In this case, the second housing 130 may include a second opening 134 formed to communicate with the accommodation space 132 so that the gripper 122 accommodated in the accommodation space 132 may move from the accommodation space 132 to the outside of the second housing 130.

In this case, the second opening 134 may serve as a working channel through which the bent portion of the flexible manipulator 124 that bends in one direction along with the gripper 122 passes when the drive cables 124d are manipulated.

Furthermore, the second opening 134 may be formed in the second housing 130 to be positioned in the same direction as the first opening 116 when the first opening 116 formed in the first housing 110 is provided on the lateral side of the housing body 112.

Through this, even though the auxiliary cap 100 for an endoscope according to an example embodiment of the present invention is coupled to the tip of a conventional side-view endoscopic device 10, the bent portion 20a of the forceps 20 may protrude through the first opening 116 in a lateral direction of the first housing 110, and the bent portion of the flexible manipulator 124 bent in one direction, including the gripper 122, may protrude laterally of the second housing 130 through the second opening 134.

Accordingly, the user may easily assist the procedure of the forceps 20, facilitated by the gripper 122.

As a result, the user can freely adjust the position of the gripper 122 that passes through the second opening 134 and is exposed to the outside of the second housing 130 by manipulating the drive cables 124d as shown in FIG. 5.

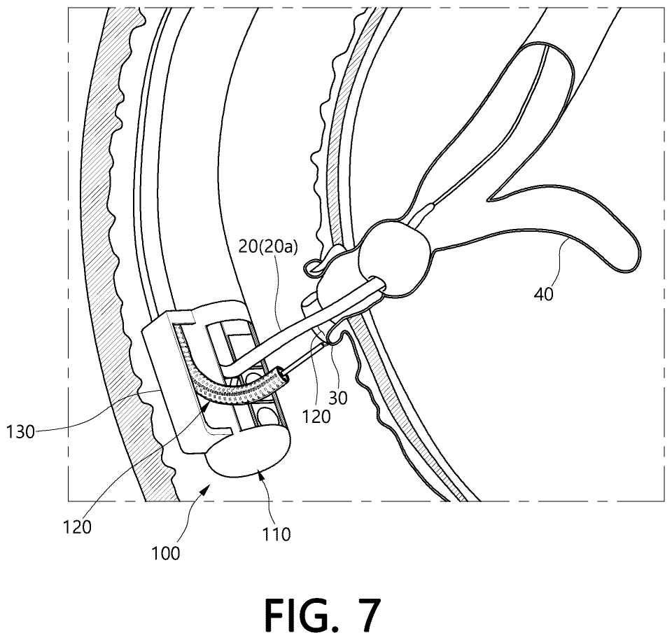

Accordingly, the user may easily enter the bent portion 20a of the forceps 20 into an entry hole 30 of an organ 40 by widening the entry hole 30 of the organ 40 or moving the entry hole 30 of the organ 40 toward the bent portion 20a of the forceps 20 by manipulating the gripper 122, as shown in FIG. 7.

Accordingly, the auxiliary cap 100 for an endoscope according to an example embodiment of the present invention can be combined with the tip of an existing side-view endoscopic device 10 to easily adjust the direction of entry of forceps 20, thereby reducing the difficulty of the procedure.

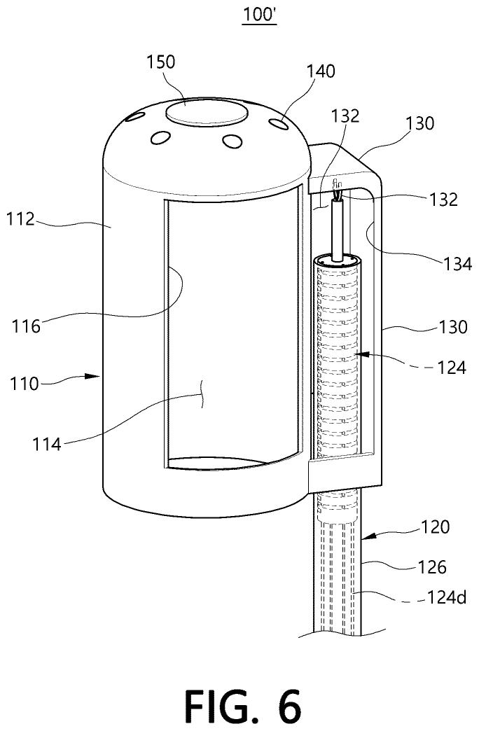

Meanwhile, an auxiliary cap 100′ for an endoscope according to an example embodiment of the present invention may further include an auxiliary light source 140 and an auxiliary camera module 150 provided at the tip of the first housing 110 as shown in FIG. 6.

Here, the auxiliary cap 100′ for an endoscope shown in FIG. 6 is identical to the auxiliary cap 100 for an endoscope described above except for the auxiliary light source 140 and the auxiliary camera module 150, so a detailed description thereof will be omitted.

Such auxiliary light source 140 and auxiliary camera module 150 may be provided at the tip of the first housing 110 to enable the user to secure a forward field of vision.

Here, the forward field of vision may be a field of vision in a direction parallel to the longitudinal direction of the main body 12 from the tip of the first housing 110, and the auxiliary light source 140 and the auxiliary camera module 150 may be supplied with electric power from the outside through a separate cable (not shown) and may provide an image acquired through the auxiliary camera module 150 to an external display device (not shown).

As described above, if the auxiliary cap 100 for an endoscope according to an example embodiment of the present invention is coupled to the tip of the side-view endoscopic device 10, light provided from the light source 14 of the main body 12 is emitted only to the lateral side of the first housing 110 through the first opening 116, so there is a problem in that the user cannot secure a forward field of vision.

However, if the auxiliary cap 100′for an endoscope according to an example embodiment of the present invention includes the auxiliary light source 140 and the auxiliary camera module 150 provided at the tip of the first housing 110, even though the auxiliary cap 100′ for an endoscope according to an example embodiment of the present invention is coupled to the tip of a side-view endoscopic device, the user can secure a forward field of vision, facilitated by the auxiliary light source 140 and the auxiliary camera module 150 provided at the tip of the first housing 110.

Accordingly, the user can secure a forward field of vision, facilitated by the auxiliary light source 140 and auxiliary camera module 150 of the auxiliary cap 100′for an endoscope according to an example embodiment of the present invention, thereby reducing the difficulty of the procedure.

That is, when the user inserts the tip of an endoscopic device into the interior of an organ to the position of the organ for surgery or procedure using a side-view endoscopic device, the entry path of the tip of the endoscopic device can be easily secured through the image acquired through the auxiliary light source 140 and the auxiliary camera module 150, so the difficulty of the procedure can be reduced.

In the drawing, the auxiliary light source 140 and the auxiliary camera module 150 are illustrated and described as being provided at the tip of the first housing 110, but the present invention is not limited thereto, and they may be provided at the tip of the second housing 130.

Although example embodiments of the present invention have been described, the idea of the present invention is not limited to the embodiments set forth herein. Those of ordinary skill in the art who understand the idea of the present invention may easily propose other embodiments through supplement, change, removal, addition, etc. of elements within the same idea, but the embodiments will be also within the idea scope of the present invention.

1. An auxiliary cap for an endoscope, the auxiliary cap being coupled to an endoscopic device comprising a light source, a forceps hole, and a camera module, and the auxiliary cap comprising:
a first housing comprising a housing body, which is formed in the shape of an enclosure and has an internal space with an open lower portion so that the housing body can be coupled to the tip of the endoscopic device, and a first opening, which has a predetermined area and passes through the housing body so as to be located at a position corresponding to the light source, the forceps hole, and the camera module when the housing body and the endoscopic device are coupled;
an auxiliary procedure member comprising a flexible manipulator, which is provided so that a portion of the length can be bent in one direction by external force transmitted through a drive cable, and a gripper, which is provided on the end side of the flexible manipulator so as to be able to assist a procedure performed using forceps; and
a second housing formed on one side of the first housing so as to form an accommodation space for accommodating a portion, including the gripper, of the entire length of the auxiliary procedure member.

2. The auxiliary cap for an endoscope of claim 1, wherein the endoscopic device is a side-view endoscopic device in which the light source, the forceps hole, and the camera module are provided on the lateral side of a tip of a main body, and wherein the first opening is formed to be positioned on the lateral side of the housing body.

3. The auxiliary cap for an endoscope of claim 1, wherein the second housing comprises a second opening formed to communicate with the accommodation space so that the gripper accommodated in the accommodation space is able to move from the accommodation space to the outside of the second housing.

4. The auxiliary cap for an endoscope of claim 3, wherein the second opening is formed in the second housing to be positioned in the same direction as the first opening.

5. The auxiliary cap for an endoscope of claim 1, wherein the flexible manipulator comprises a hollow body having a predetermined length, a plurality of first cut-out portions formed circumferentially along the body, a plurality of second cut-out portions formed circumferentially along the body without being connected to the first cut-out portions, and a plurality of drive cables, each of which is fixed to an end side of the body.

6. The auxiliary cap for an endoscope of claim 1, wherein the auxiliary cap for an endoscope further comprises an auxiliary light source and an auxiliary camera module provided at the tip of the first housing.
